(19) United States
(12) Patent Application Publication
(10) Pub. No.: US 2014/0025144 A1
(43) Pub. Date: Jan. 23, 2014

(54) ICY EYEWEAR

(71) Applicant: Cindy Robin Ragan, Wilton Manors, FL (US)

(72) Inventor: Cindy Robin Ragan, Wilton Manors, FL (US)

(21) Appl. No.: 13/944,827

(22) Filed: Jul. 17, 2013

Related U.S. Application Data
(60) Provisional application No. 61/742,060, filed on Jul. 20, 2012.

(51) Int. Cl. A61F 7/10

(52) U.S. Cl. CPC A61F 7/10
USPC 607/109

(57) ABSTRACT

An icy eyewear apparatus comprising: a front sinusoidal rim having a convex section in its center, and two concave sections, such that, when the icy eyewear apparatus is worn by the user, each concave section mirrors the shape of the bottom of the eye of the user from the respective side and each concave section lies proximate to the bottom of the respective eye, such that the user's vision is not obstructed; two temple pieces, one temple piece associated with each side, such that, when the icy eyewear apparatus is worn by the user, the two temple pieces hold the icy eyewear apparatus in place by securing it to the ears of the user; and, two icy packets, one icy packet attached to each concave section of the front sinusoidal rim, such that, the icy packets rest on the swollen areas below the eyes of the user.

### CROSS-REFERENCE TO RELATED APPLICATIONS

This application claims the benefit of U.S. Provisional Application No. 61/742,060, filed Jul. 20, 2012, which is hereby incorporated by reference, to the extent that it is not conflicting with the present application.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

1. Field of the Invention

The invention relates to an apparatus and method for reducing puffiness of the area below the eyes.

2. Description of the Related Art

Puffiness of the area below the eyes is a common problem to many middle aged and elderly people. While there are currently solutions to puffy eyes, based on using cold creams, cold compresses, cucumber, ice or ice gel packets, they appear to require the user to lie down during use. This is highly inconvenient as the user is being unable to do anything else during that time. Other devices that are offered to solve this problem go around the back of the head, messing up user's hair, and/or preventing the user from working on her hair while wearing the respective devices. Yet other devices cover the eyes completely, such that the user cannot see. This solution is obviously highly inconvenient as well as it renders the users completely incapacitated and prevents them from doing other things, such as getting ready for work, a party, a date, a meeting, and so on. Most people do not have the time or the patience to use the above solutions. Thus, there is a need for a new and improved apparatus and method that overcome the deficiencies of the current apparatuses and methods.

The problems and the associated solutions presented in this section could be or could have been pursued, but they are not necessarily approaches that have been previously conceived or pursued. Therefore, unless otherwise indicated, it should not be assumed that any of the approaches presented in this section qualify as prior art merely by virtue of their presence in this section of the application.

### BRIEF SUMMARY OF THE INVENTION

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

In one exemplary embodiment, an icy eyewear apparatus is provided, which is wearable in a way similar to a pair of glasses (i.e., nose and ears support) and such that it does not interfere with wearer's ability to see, and which includes an icy packet for each of the areas below one's eye, for reducing puffiness in those areas. Thus, an advantage is that a user can reduce the “bags” under her eyes, and thus, her appearance, while getting ready for work, a party, a meeting, etc, without interrupting her normal routine during that time period.

The above embodiment and advantages, as well as other embodiments and advantages, will become apparent from the ensuing description and accompanying drawings.

### BRIEF DESCRIPTION OF THE DRAWINGS

For exemplification purposes, and not for limitation purposes, embodiments of the invention are illustrated in the figures of the accompanying drawings, in which:

FIG. 1 illustrates schematically a front view of a user wearing the icy eyewear apparatus disclosed herein, according to an embodiment.

FIG. 2 illustrates schematically a side view of a user wearing the icy eyewear apparatus.

FIGS. 3a-d illustrate a side-perspective, a top, a front and a side view, respectively, of the frame of the icy eyewear apparatus disclosed herein, according to another embodiment.

FIGS. 4a-c illustrate a front, a side and a bottom view, respectively, of the ice packet of the icy eyewear apparatus disclosed herein.

### DETAILED DESCRIPTION OF THE PREFERRED EMBODIMENTS

What follows is a detailed description of the preferred embodiments of the invention in which the invention may be practiced. Reference will be made to the attached drawings, and the information included in the drawings is part of this detailed description. The specific preferred embodiments of the invention, which will be described herein, are presented for exemplification purposes, and not for limitation purposes. It should be understood that structural and/or logical modifications could be made by someone of ordinary skills in the art without departing from the scope of the invention. Therefore, the scope of the invention is defined by the accompanying claims and their equivalents.

For the following description, it can be assumed that most correspondingly labeled structures across the figures (e.g., 100 and 300, etc.) possess the same characteristics and are subject to the same structure and function. If there is a difference between correspondingly labeled elements that is not pointed out, and this difference results in a non-corresponding structure or function of an element for a particular embodiment, then that conflicting description given for that particular embodiment shall govern.

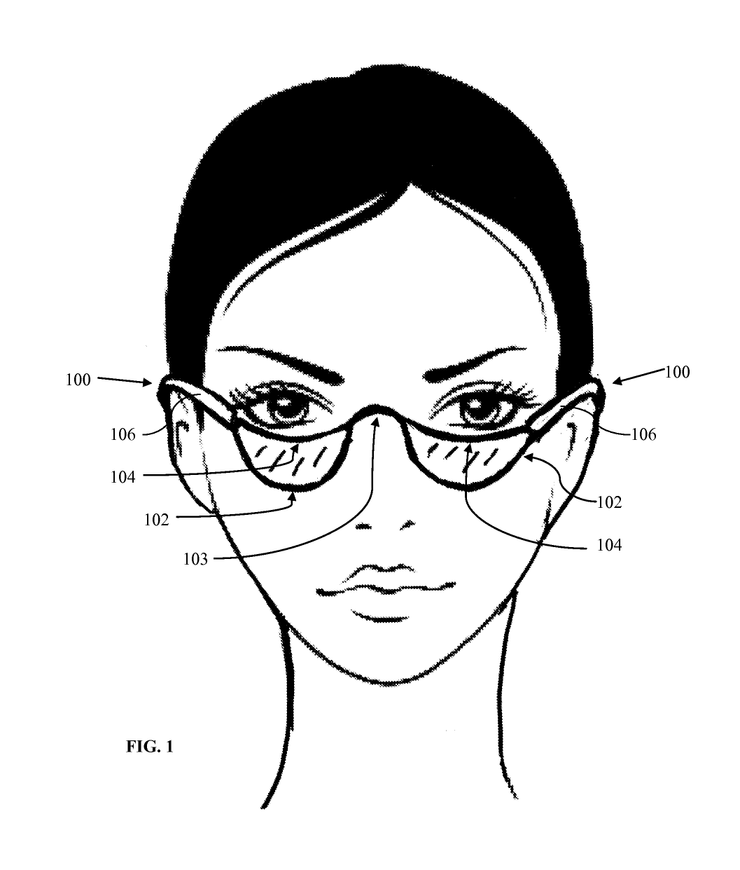

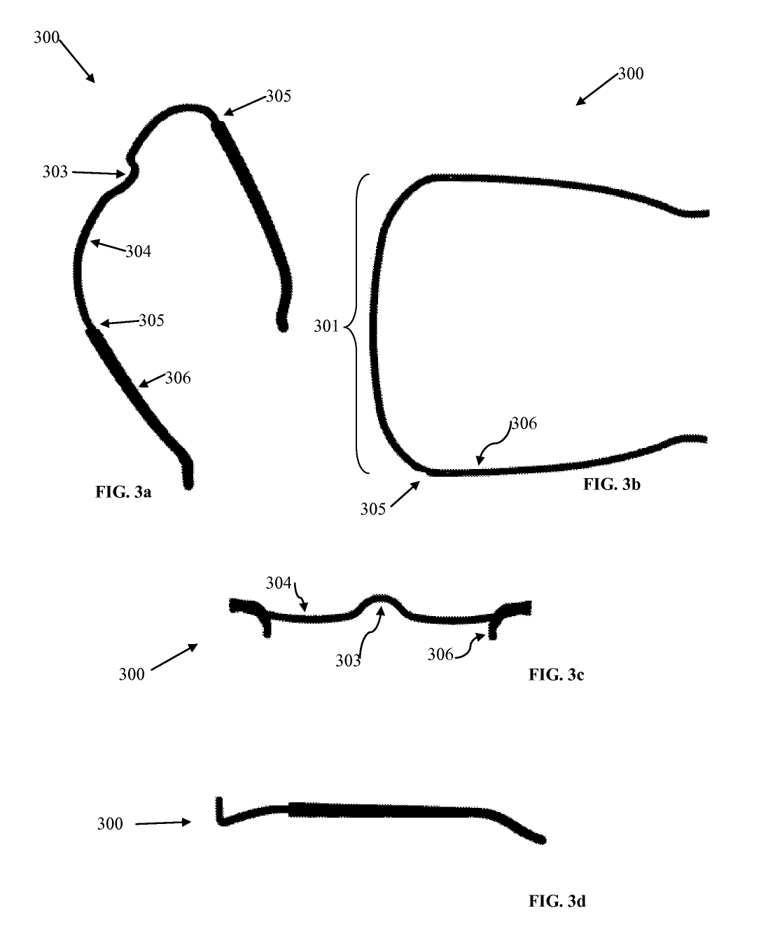

FIG. 1 illustrates schematically a front view of a user wearing the icy eyewear apparatus disclosed herein, according to an embodiment. As shown, the icy eyewear apparatus has two main components, the frame 100 (300 in FIGS. 3a-c) and the icy packets 102. The frame 100 includes the temple pieces 106, one on each side, and a front rim (301 in FIG. 3b). The front rim is typically a one-piece element shaped sinusoidally, such that it has two concave sections 104 (304 in FIGS. 3a and 3c), one on each side, and one convex section 103 (303 in FIGS. 3a and 3c) between the two concave sections. The convex section 103 is shaped such that it fits on a typical nose of a user/wearer such that it supports most of the weight of the icy eyewear apparatus. As shown, the concave sections 104 are shaped such as they mirror the shape of the eyes of a typical user, namely, the bottom of the eye, and lie away from the bottom of the eye only as necessary so that the user's vision is not obstructed at all when the icy eyewear apparatus is worn by the user.

The icy eyewear apparatus may be manufactured in various sizes to account for differences in users' eye sizes or shapes, facial characteristics, weight, and so on.

The temple pieces 106 (306 in FIGS. 3a-c) have the shape and function which are similar with the temples of eyeglasses or sunglasses. They help hold the icy eyewear apparatus in place. Referring to FIGS. 3a and 3b, at the two junctions 305 between the temple pieces 306 and the front rim 301, a hinge may be used such as the temple pieces 106 may be conveniently folded inward during storage or cooling of the icy eyewear apparatus.

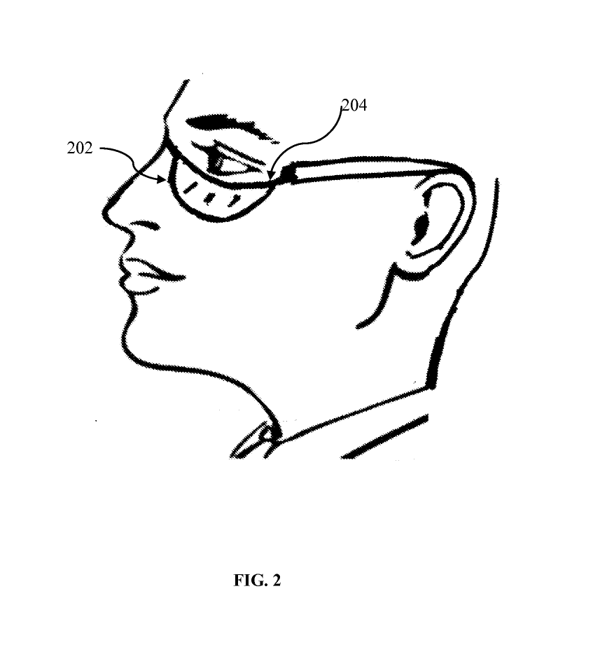

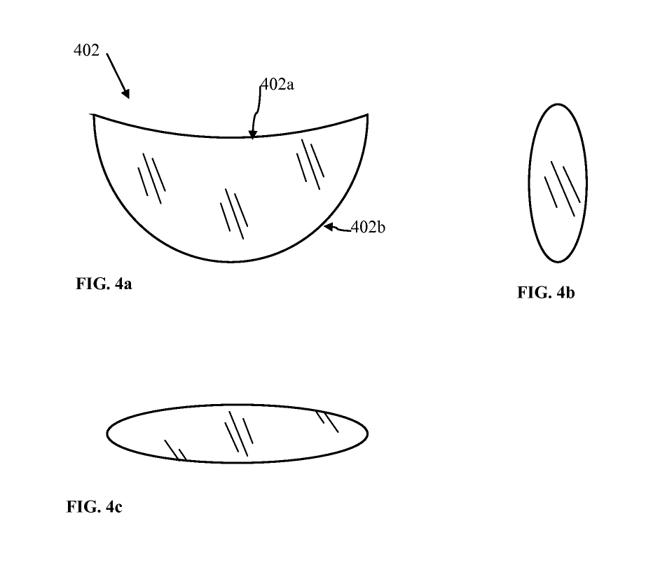

The icy packets 102 (402 in FIG. 4a) may have various shapes. However, the preferred shape may be one that resembles a moon, with a convex bottom edge 402b and a concave upper edge 402a. The curvature of the concave upper edge 402a corresponds preferably with the curvature of the concave sections 104, such that the upper edge 402a may be used to snuggly attach, removably or irremovably, the ice packets 102 to the concave sections 104 of the frame 100. This preferred requirement for the curvature of the concave upper edge 402a also ensures that it corresponds with the typical shape of the eye of the user/wearer. The preferred moon shape of the icy packets 102 mirrors the typical shape of the swollen area under one's eyes, which the icy packets are intended to cover and minimize as it will described in more details hereinafter. Various means known in the art may be used to attach/fasten the icy packets 102 (202 in FIG. 2) to the concave sections 104 (204 in FIG. 2) of the frame 100. Such means may include glue, Velcro™, elastic strap, and so on. Probably the main consideration when choosing one attachment means or another is the degree of comfort to the user.

The icy packets 102 may be small plastic bags filled with a gel similar to the one used in typical soft freeze gel packs, used for example to reduce pain from athletic or work related injuries. A gel with a higher degree of softness may be preferred such that the icy packets 102 conform better to the swollen area under the eye. Referring to FIGS. 4a-c, the icy packets 402 may be approximately one inch tall, two inches wide and approximately one half to one inch thick.

Referring now to FIGS. 3a-d, the frame 300 may be made of various materials such as metal, plastic, wood, and so on. However, the preferred material for the frame 300 is a plastic that does not freeze and that does not considerably retain the cold (e.g., polycarbonate). The purpose for using such a plastic is to increase the comfort of the user/wearer while the icy eyewear apparatus is worn, by ensuring that only the ice/gel packets are cold.

### Method of Use

First, a user would place the icy eyewear apparatus in the freezer so that the icy packets 102 are cold when the icy eyewear apparatus is needed at a later time. Again, the icy eyewear apparatus may be constructed as explained earlier, such that the temple pieces 106 are foldable inward so that the apparatus does not take too much space in the freezer. Also, as described earlier, the icy packets 102 may be irremovably attached to the frame 100. Thus, in that instance, the whole apparatus, that is, the frame 100 with the icy packets 102 attached, will be place in the freezer. Alternatively, again, the icy packets 102 may be removably attached to the frame 100. Thus, a user may choose to detach the icy packets 102 from the frame 100 before placing the icy packets in the freezer for later use. This may have the advantage that the frame 100 will not be inconveniently cold when the apparatus is in worn by the user. The user may simply reattach the cold icy packets 102 to the frame 100, which may be comfortably at room temperature, before using the icy eyewear apparatus.

Next, when the user wishes to improve her appearance for, for example, a special occasion, by decreasing the puffiness, or “bags,” under her eyes, she may wear the icy eyewear apparatus with the cold icy packets as described earlier. Again, one benefit of the disclosed icy eyewear apparatus is that the user may wear it while going on with her normal routine (e.g., getting ready for the special occasion). This is because her vision is not impaired at all and because the icy packets will remain securely in place, over the puffy area under her eyes, to do their job of decreasing the puffiness of that area. Another benefit of the icy eyewear apparatus is that it just treats the bottom of the eyes; therefore, the apparatus does not affect women's eye make-up, which is a “big deal” to women. It has been observed that, by wearing the icy eyewear apparatus disclosed herein, in only about ten minutes, the puffiness under the eyes is considerably reduced. The special occasion may be a party, a meeting, a date, and so on, or simply going to work or in public (e.g., shopping).

It may be advantageous to set forth definitions of certain words and phrases used in this patent document. The terms “include” and “comprise,” as well as derivatives thereof, mean inclusion without limitation. The term “or” is inclusive, meaning and/or. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like.

Although specific embodiments have been illustrated and described herein for the purpose of disclosing the preferred embodiments, someone of ordinary skills in the art will easily detect alternate embodiments and/or equivalent variations, which may be capable of achieving the same results, and which may be substituted for the specific embodiments illustrated and described herein without departing from the scope of the invention. Therefore, the scope of this application is intended to cover alternate embodiments and/or equivalent variations of the specific embodiments illustrated and/or described herein. Hence, the scope of the invention is defined by the accompanying claims and their equivalents. Furthermore, each and every claim is incorporated as further disclosure into the specification and the claims are embodiment(s) of the invention.

What is claimed is:

1. An icy eyewear apparatus comprising: a front sinusoidal rim having a convex section in its center, which, when the icy eyewear apparatus is worn by a user, rests on the nose of the user, such that it supports most of the weight of the icy eyewear apparatus, and two concave sections, one on each side, left and right, of the convex section, such that, when the icy eyewear apparatus is worn by the user, each concave section corresponds to the shape of the bottom of the eye of the user from the respective side and each concave section lies proximate to the bottom of the respective eye, such that the user's vision is not obstructed; two temple pieces, one temple piece associated with each side, left and right, of the front sinusoidal rim, such that, together, when the icy eyewear apparatus is worn by the user, the two temple pieces hold the icy eyewear apparatus in place by securing it to the ears of the user; and, two icy packets, one icy packet attached to each concave section of the front sinusoidal rim, such that, when the icy eyewear apparatus is worn by the user, the icy packets rest on the swollen areas below the eyes of the user.

2. The icy eyewear apparatus of claim 1, wherein the icy packets are filled with ice.

3. The icy eyewear apparatus of claim 1, wherein the icy packets are filled with a gel, such that the icy packets remain soft after exposing them to water freezing temperature.

4. The icy eyewear apparatus of claim 1, wherein the icy packets are removably attached to the front sinusoidal rim.

5. The icy eyewear apparatus of claim 1, wherein the icy packets have a moon shape such that they closely fit the shape of the swollen areas below the eyes of the user.

6. A method for reducing swelling under a person's eyes by using the icy eyewear apparatus of claim 1 and comprising the steps of: placing at least a portion of the icy eyewear apparatus in a home freezer; and, the person wearing the icy eyewear apparatus for a predetermined period of time, while attending to her usual routine, and without affecting the person's eye make-up.

7. The method of claim 6, wherein, only the icy packets of the icy eyewear apparatus are placed in the home freezer.

8. The method of claim 6, wherein, the predetermined period of time is ten minutes.
